(19) United States
(12) Patent Application Publication
(10) Pub. No.: US 2025/0209591 A1
(43) Pub. Date: Jun. 26, 2025

(54) ULTRASONIC IMAGING METHOD, APPARATUS, DEVICE, AND STORAGE MEDIUM

(71) Applicant: WUHAN UNITED IMAGING HEALTHCARE CO., LTD., Wuhan (CN)

(72) Inventor: Tong Li, Hubei (CN)

(21) Appl. No.: 19/001,407

(22) Filed: Dec. 24, 2024

(30) Foreign Application Priority Data
Dec. 25, 2023 (CN) 202311800106.5

(51) Int. Cl. G06T 7/00; A61B 8/00

(52) U.S. Cl. CPC G06T 7/0002; A61B 8/5207; G06T 2207/10132; G06T 2207/20084

(57) ABSTRACT

An ultrasonic imaging method disclosed in the present disclosure includes: acquiring a plurality sets of channel data of a target part; performing phase compensations on the plurality sets of the channel data to obtain a plurality sets of compensated channel data corresponding to the plurality sets of the channel data respectively; and obtaining an ultrasonic image of the target part according to the plurality sets of the compensated channel data.

### CROSS-REFERENCE TO RELATED APPLICATIONS

The present application claims priority to Chinese patent application No. 202311800106.5, filed on Dec. 25, 2023, and entitled “ULTRASONIC IMAGING METHOD, APPARATUS, DEVICE, AND STORAGE MEDIUM”, the entire content of which is incorporated herein by reference.

### TECHNICAL FIELD

The present disclosure relates to the field of oral ultrasonic imaging technologies, and in particular, to an ultrasonic imaging method, apparatus, device, and storage medium.

### BACKGROUND

Ultrasonic imaging has become one of the most widely used diagnostic tools in clinical practice due to its many advantages such as non-invasiveness, real-time nature, ease of operation and low price.

In the related art, ultrasonic waves are emitted to a target tissue of a target object by an ultrasonic probe, ultrasonic echoes reflected by the target tissue are received, and ultrasonic echoes are beam synthesized to obtain ultrasonic echo channel signals. Finally, an ultrasonic image is obtained by performing an imaging process on the ultrasonic echo channel signals.

However, in the related art, there is a technical problem of poor quality of the ultrasonic image.

### SUMMARY

The present disclosure provides an ultrasonic imaging method, apparatus, device, and storage medium.

In a first aspect, the present disclosure provides an ultrasonic imaging method, including: acquiring a plurality sets of channel data of a target part, performing phase compensations on the plurality sets of the channel data to obtain a plurality sets of compensated channel data corresponding to the plurality sets of the channel data respectively, and obtaining an ultrasonic image of the target part according to the plurality sets of the compensated channel data.

In an embodiment, performing the phase compensations on the plurality sets of the channel data to obtain the plurality sets of the compensated channel data corresponding to the plurality sets of the channel data includes: inputting the plurality sets of the channel data into a pre-trained phase compensation model to obtain a plurality sets of phase compensation data for the corresponding channel data, and determining the plurality sets of the compensated channel data according to the corresponding phase compensation data and the corresponding channel data.

In an embodiment, the ultrasonic imaging method further includes training the phase compensation model. Training the phase compensation model includes: acquiring sample channel data, the sample channel data including training channel data and validation channel data, training a neural network model using the training channel data to obtain an initial phase compensation model, and adjusting model parameters of the initial phase compensation model using the validation channel data to obtain the phase compensation model.

In an embodiment, the sample channel data further includes test channel data, and the method further includes evaluating a generalization ability of the phase compensation model based on the test channel data.

In an embodiment, training the neural network model using the training channel data to obtain the initial phase compensation model includes: inputting the training channel data into the neural network model to obtain training phase compensation data corresponding to the training channel data, determining a compensation loss according to the training phase compensation data, and adjusting the model parameters of the neural network model based on the compensation loss until the compensation loss is less than a preset threshold, to obtain the initial phase compensation model.

In an embodiment, determining the compensation loss according to the training phase compensation data includes: acquiring target phase compensation data corresponding to the training channel data, and determining the compensation loss according to the training phase compensation data and the target phase compensation data.

In an embodiment, the ultrasonic imaging method further includes: determining parameter values of at least one of phase distortion parameters according to the plurality sets of the compensated channel data, and evaluating a quality of the ultrasonic image according to the parameter values.

In some embodiments, the phase distortion parameters include at least one of distortion intensity, correlation length of phase distortion, energy level fluctuation, and coherence coefficient.

In some embodiments, the phase distortion parameters include a coherence coefficient. Determining the parameter values of the at least one of the phase distortion parameters according to the plurality sets of the compensated channel data includes: performing beam synthesis on the plurality sets of the compensated channel data to obtain a plurality sets of beam synthesized data, obtaining a plurality sets of mean value data of the plurality sets of the beam synthesized data, based on the plurality sets of the beam synthesized data, and determining the parameter values of the coherence coefficient based on the plurality sets of the beam synthesized data and the plurality sets of the mean value data.

In some embodiments, the phase distortion parameters include distortion intensity. Determining the parameter values of the at least one of the phase distortion parameters according to the plurality sets of the compensated channel data includes: performing beam synthesis on the plurality sets of the compensated channel data to obtain a plurality sets of beam synthesized data, and acquiring delay error data from the beam synthesized data, and determining parameter values of the distortion intensity based on the delay error data.

In some embodiments, evaluating the quality of the ultrasonic image according to the parameter values includes: comparing each of the parameter values with a corresponding preset parameter value threshold range, and determining that the quality of the ultrasonic image is qualified on a condition that each of the parameter values falls within the corresponding preset parameter value threshold range.

In some embodiments, obtaining the ultrasonic image of the target part according to the plurality sets of the compensated channel data includes: performing beam synthesis on the plurality sets of the compensated channel data to obtain image data, and obtaining the ultrasonic image according to the image data.

In a second aspect, the embodiments of the present disclosure further provide a computer device. The computer device includes a memory and a processor, and the memory stores a computer program. The processor, when executing the computer program, performs an ultrasonic imaging method, which includes: acquiring a plurality sets of channel data of a target part, performing phase compensations on the plurality sets of the channel data to obtain a plurality sets of compensated channel data corresponding to the plurality sets of the channel data respectively, and obtaining an ultrasonic image of the target part according to the plurality sets of the compensated channel data.

In some embodiments, the processor, when executing the computer program, performs the phase compensations on the plurality sets of the channel data to obtain a plurality sets of compensated channel data corresponding to the plurality sets of the channel data, which includes: inputting the plurality sets of the channel data into a pre-trained phase compensation model to obtain a plurality sets of phase compensation data for the corresponding channel data, and determining the plurality sets of the compensated channel data according to the corresponding phase compensation data and the corresponding channel data.

In some embodiments, the processor, when executing the computer program, trains the phase compensation model, which includes: acquiring sample channel data, the sample channel data including training channel data and validation channel data, training a neural network model using the training channel data to obtain an initial phase compensation model, and adjusting model parameters of the initial phase compensation model using the validation channel data to obtain the phase compensation model.

In some embodiments, the processor, when executing the computer program, trains the neural network model using the training channel data to obtain an initial phase compensation model, which includes: inputting the training channel data into the neural network model to obtain training phase compensation data corresponding to the training channel data, determining a compensation loss according to the training phase compensation data, and adjusting the model parameters of the neural network model based on the compensation loss until the compensation loss is less than a preset threshold, to obtain the initial phase compensation model.

In some embodiments, when executing the computer program, the processor further implements: determining parameter values of at least one of phase distortion parameters according to the plurality sets of the compensated channel data, and evaluating a quality of the ultrasonic image according to the parameter values.

In some embodiments, the processor, when executing the computer program, determines the parameter values of the at least one of phase distortion parameters according to the plurality sets of the compensated channel data, which includes: performing beam synthesis on the plurality sets of the compensated channel data to obtain a plurality sets of beam synthesized data, obtaining a plurality sets of mean value data of the plurality sets of the beam synthesized data, based on the plurality sets of the beam synthesized data, and determining parameter values of coherence coefficient based on the plurality sets of the beam synthesized data and the plurality sets of the mean value data, the phase distortion parameters including the coherence coefficient.

In some embodiments, the processor, when executing the computer program, evaluates the quality of the ultrasonic image according to the parameter values, which includes: comparing each of the parameter values with a corresponding preset parameter value threshold range, and determining that the quality of the ultrasonic image is qualified on a condition that each of the parameter values falls within the corresponding preset parameter value threshold range.

In a third aspect, the embodiments of the present disclosure further provide a non-transitory computer-readable storage medium. The computer-readable storage medium stores a computer program, and the computer program, when executed by a processor, causes the processor to perform an ultrasonic imaging method. The ultrasonic imaging method includes: acquiring a plurality sets of channel data of a target part, performing phase compensations on the plurality sets of the channel data to obtain a plurality sets of compensated channel data corresponding to the plurality sets of the channel data respectively, and obtaining an ultrasonic image of the target part according to the plurality sets of the compensated channel data.

### BRIEF DESCRIPTION OF THE DRAWINGS

In order to describe the technical solutions in the embodiments of the present application or the conventional technology more clearly, the following will briefly introduce the accompanying drawings required for describing the embodiments or the conventional technology. Apparently, the accompanying drawings in the following description are merely embodiments of the present disclosure, and for a person of ordinary skill in the art, other drawings can be obtained based on the disclosed drawings without creative efforts.

FIG. 1 is a schematic diagram illustrating an internal configuration of a computer device according to an embodiment of the present disclosure.

FIG. 2 is a flowchart of an ultrasonic imaging method in some embodiments of the present disclosure.

FIG. 3 is a flowchart of determining compensated channel data in some embodiments of the present disclosure.

FIG. 4 is a flowchart of training a phase compensation model in some embodiments of the present disclosure.

FIG. 5 is a flowchart of training an initial phase compensation model in some embodiments of the present disclosure.

FIG. 6 is a flowchart of evaluating a quality of an ultrasonic image in some embodiments of the present disclosure.

FIG. 7 is a flowchart of determining parameter values in some embodiments of the present disclosure.

FIG. 8 is a flowchart of an ultrasonic imaging method in other embodiments of the present disclosure.

FIG. 9 is a schematic diagram of a configuration of an ultrasonic imaging apparatus in some embodiments of the present disclosure.

### DETAILED DESCRIPTION OF THE EMBODIMENTS

To make the purpose, technical solution, and advantages of the present disclosure more clear and understandable, the following detailed description is given in conjunction with the drawings and embodiments. It should be understood that the specific embodiments described herein are merely illustrative to explain the present disclosure and are not intended to limit the scope of the present disclosure.

An ultrasonic imaging method provided in embodiments of the present disclosure can be applied to a computer device. The computer device can be a server, and its schematic diagram of an internal configuration can be shown in FIG. 1. The computer device includes a processor, a memory, an input/output interface (Input/Output, referred to as I/O) and a communication interface. The processor, the memory and the input/output interface are connected through a system bus, and the communication interface is connected to the system bus through the input/output interface. The processor of the computer device is configured to provide computing and control capabilities. The memory of the computer device includes a non-transitory storage medium and an internal memory. The non-transitory storage medium stores an operating system, a computer program and a database. The internal memory provides an environment for running of the operating system and the computer program in the non-transitory storage medium. The database of the computer device is configured to store ultrasonic imaging data. The input/output interface of the computer device is configured to exchange information between the processor and an external device. The communication interface of the computer device is configured to communicate with an external terminal through a network connection. The computer program is executed by the processor to implement an ultrasonic imaging method. Those skilled in the art may understand that a structure shown in FIG. 1 is only a block diagram of a partial structure related to the solution of the present disclosure, and constitutes no limitation on the computer device to which the solution of the present disclosure is applied. The specific computer device may include more or fewer components than those shown in the drawing, or some components may be combined, or a different component deployment may be used.

Ultrasonic imaging has become one of the most widely used diagnostic tools in clinical practice due to its many advantages such as non-invasiveness, real-time nature, ease of operation and low price.

In the related art, ultrasonic waves are emitted to a target tissue of a target object by an ultrasonic probe, ultrasonic echoes reflected by the target tissue are received, and ultrasonic echoes are beam synthesized to obtain ultrasonic echo channel signals. Finally, an ultrasonic image is obtained by performing an imaging process on the ultrasonic echo channel signals.

Beam synthesis refers to superimposing signals from different channels to improve the signal-to-noise ratio and suppress side lobes. The delay in beam synthesis is crucial, and inaccurate delay will reduce the signal-to-noise ratio of images. If phase differences of the delay of channels cannot be effectively superimposed, it will cause focusing errors, and reduce imaging contrast and spatial resolution.

However, due to the non-uniformity of the sound velocity in human tissue, phase distortion is almost inevitable. Therefore, certain measures need to be taken to compensate the phase of the channel signals to improve the quality of ultrasonic imaging.

Based on this, an ultrasonic imaging method is provided in the present disclosure. Phase compensations are performed on a plurality sets of channel data of a target part to obtain compensated channel data, i.e., image reconstructions can be performed according to the compensated channel data. In this way, the channel data with phase distortion is restored to channel data without phase distortion, so that the phase difference of the delay of the channels can be effectively superimposed, which improves the focusing quality, thereby improving the image contrast and spatial resolution, i.e., improving the quality of the ultrasonic image.

It should be noted that the beneficial effects or the solved technical problems of the embodiments of the present disclosure are not limited to this one, but may also include other implicit or related problems. For details, refer to the description of the following embodiments.

The following specific embodiments are used to describe in detail the technical solution of the present disclosure and how the technical solution of the present disclosure solves the above-mentioned technical problems. The following specific embodiments can be combined with each other, and the same or similar concepts or processes may not be repeated in some embodiments. The embodiments of the present disclosure will be described below in connection with the accompanying drawings.

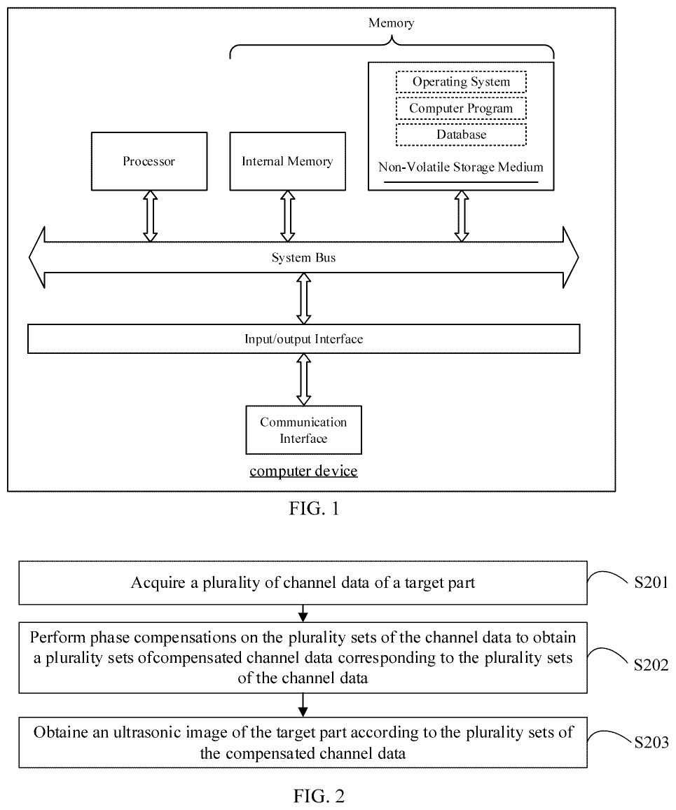

In some exemplary embodiments, as shown in FIG. 2, an ultrasonic imaging method is provided, which is described by taking the method applied to a computer device as an example, and includes the following steps of S201 to S203.

In S201, a plurality sets of channel data of a target part are acquired.

In the embodiments of the present disclosure, the target part refers to a part of the target object that needs to be imaged using ultrasound. The types of the target part may include but are not limited to small organs, nerves, a heart, an abdomen, and muscle bones.

The channel data may refer to data corresponding to an array element in an ultrasonic probe before beam synthesis is performed, for example, the channel data may be a radio frequency signal before demodulation, or a baseband signal after demodulation, etc. One array element of the ultrasonic probe outputs one channel data correspondingly.

In the present disclosure, the channel data of the target part can be acquired in real time by transmitting ultrasonic waves to the target part by the ultrasonic probe and receiving ultrasonic echoes returned from the target part. Alternatively, the channel data of the target part stored in advance can be acquired from a storage device.

In S202, phase compensations are performed on the plurality sets of the channel data to obtain a plurality sets of compensated channel data corresponding to the plurality sets of the channel data respectively.

The phase compensation can be understood as restoring the channel data with phase distortion to the channel data without phase distortion. The compensated channel data refers to the channel data that has been undergone phase compensation.

In some embodiments, after acquiring the plurality sets of channel data of the target part, the computer device can input the plurality sets of the channel data of the target part into a pre-trained compensation model, to obtain the plurality sets of compensated channel data corresponding to the plurality sets of the channel data by analyzing the plurality sets of the channel data by the compensation model.

The compensation model may be a pre-trained neural network model. Optionally, the compensation model may be established using a network model such as an error back propagation neural network, a recurrent neural network, a deep neural network or a convolutional neural network.

In S203, an ultrasonic image of the target part is obtained according to the plurality sets of the compensated channel data.

In some embodiments of the present disclosure, phase-compensated channel data can be used for image reconstruction. Optionally, beam synthesis can be performed on the plurality sets of the compensated channel data to obtain image data, and then the ultrasonic image of the target part can be obtained according to the image data.

According to the ultrasonic imaging method provided in the embodiment of the present disclosure, the plurality sets of the channel data of the target part are acquired, and then the phase compensations is performed on the plurality sets of the channel data to obtain the plurality sets of the compensated channel data corresponding to the plurality sets of the channel data respectively. Finally, the ultrasonic image of the target part is obtained according to the plurality sets of the compensated channel data. In this method, by performing the phase compensations on the plurality sets of channel data of the target part to obtain the plurality sets of the compensated channel data, the image reconstruction can be performed according to the compensated channel data, so that the channel data with phase distortion is restored to the channel data without phase distortion, so that the phase difference of the delay of the channels can be effectively superimposed, which improves the focusing quality, thereby improving the image contrast and spatial resolution, i.e., improving the quality of the ultrasonic image.

With the extensive use of neural networks, the processing of the ultrasonic image has gradually shifted from post-processing, i.e., image analysis or feature extraction, to mid-processing in the ultrasound link, such as correlation coefficients, correlation between scan lines, or front-end processing, i.e., a processing of the channel data. In some embodiments of the present disclosure, the processing of the channel data can also be performed by a neural network. Based on this, in the following embodiment, the method of performing the phase compensations on the plurality sets of the channel data is described.

In some exemplary embodiments, as shown in FIG. 3, performing the phase compensations on the plurality sets of the channel data to obtain the plurality sets of the compensated channel data corresponding to the plurality sets of the channel data includes the following steps of S301 and S302.

In S301, the plurality sets of the channel data are input into a pre-trained phase compensation model to obtain a plurality sets of phase compensation data for the corresponding channel data.

In some embodiments of the present disclosure, the phase compensation model can be trained based on a neural network model. The types of neural network models may include but are not limited to a convolutional neural network, a generative adversarial network, a recurrent neural network, a fully connected neural network, a deep neural network, and an error back propagation neural network.

Training the phase compensation model may include: obtaining a plurality sets of historical channel data of multiple objects, and using each of the historical channel data of the multiple objects as training data; and inputting the training data into the neural network model, and training the neural network model according to preset model training parameters, until the neural network model meets a preset condition, to obtain the phase compensation model. The preset condition may be that the loss function of the neural network model is less than a preset threshold or a number of iterations of the neural network model reaches a preset number of iterations. The input of the phase compensation model is the historical channel data, and the output of the phase compensation model is the phase compensation data corresponding to each of the historical channel data. The phase compensation data refers to phase compensation value.

After obtaining the trained phase compensation model, the plurality sets of the channel data are input into the trained phase compensation model, the plurality sets of the channel data are analyzed by the phase compensation model to obtain the plurality sets of phase compensation data for the corresponding channel data. The phase compensation data can refer to a phase distortion distribution of each array element. The following formula (1) is an expression of the phase compensation data y:

$$y ( n ) = x ( n ) {e}^{jw τ ( n )} \qquad ( 1 )$$

where n refers to the array element, τ(n) refers to a delay to be compensated for a nth array element, x refers to a change intensity which varies from 0 to 1, w refers to an angular speed, and the phase wτ(n) ranges from −π/2 to π/2.

In S302, the plurality sets of the compensated channel data are determined according to the corresponding phase compensation data and the corresponding channel data.

After the phase compensation data for the corresponding channel data is obtained, the plurality sets of the channel data are fused with the phase compensation data for the corresponding channel data to obtain the plurality sets of the compensated channel data corresponding to the plurality sets of the channel data.

In the ultrasonic imaging method provided in the embodiment of the present disclosure, the plurality sets of the channel data are input into a pre-trained phase compensation model to obtain the plurality sets of phase compensation data for the corresponding channel data, and then the plurality sets of the compensated channel data are determined according to the corresponding phase compensation data and the corresponding channel data. In this method, an optional way to perform the phase compensations on the plurality sets of the channel data is provided. The phase compensation model is obtained by training the neural network model so that the plurality sets of the channel data of the target part are analyzed by the phase compensation model to obtain the plurality sets of the phase compensation data corresponding to the plurality sets of the channel data respectively, and the plurality sets of the compensated channel data can be obtained according to the corresponding phase compensation data and the corresponding channel data.

In order to obtain a more accurate phase compensation model by training, a certain amount of sample channel data can be obtained, and the sample channel data can be divided into a training set and a test set to train the neural network model. Based on this, training the phase compensation model is described in the following embodiment.

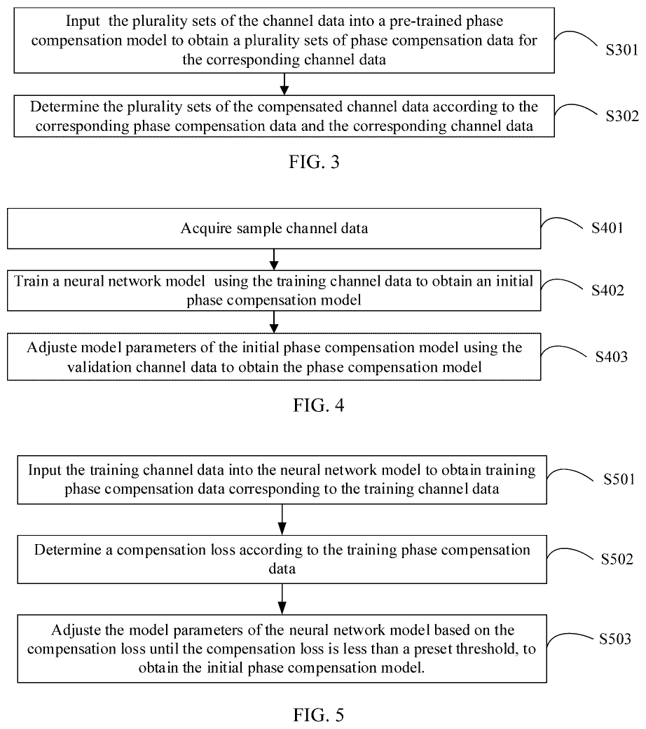

In some exemplary embodiments, as shown in FIG. 4, training the phase compensation model includes the following steps of S401 to S403.

In S401, sample channel data is acquired.

In the embodiment of the present disclosure, the sample channel data includes training channel data and validation channel data.

Exemplarily, the channel data of multiple objects may be acquired from a database storing the channel data as the sample channel data, and the sample channel data may be divided into training channel data and validation channel data in a ratio of 7:3.

In S402, a neural network model is trained using the training channel data to obtain an initial phase compensation model.

The types of the neural network model may include but are not limited to a convolutional neural network, a generative adversarial network, a recurrent neural network, a fully connected neural network, a deep neural network, and an error back propagation neural network.

Exemplarily, the training channel data is input into the neural network model for multiple iterations of training until a preset training stop condition is reached, then the network parameters of the neural network model are stopped to be adjusted to obtain a trained neural network model, i.e., the initial phase compensation model. The training stop condition may include that a number of training times reaches a preset number of times, or a training loss converges, etc.

In S403, model parameters of the initial phase compensation model are adjusted using the validation channel data to obtain the phase compensation model.

After the initial phase compensation model is obtained by training, the capability of the initial phase compensation model can be initially evaluated using validation channel data, and the model parameters of the initial phase compensation model can be adjusted in this process to obtain a final phase compensation model.

It should be noted that a portion of channel data can also be selected from the sample channel data as test channel data, and the generalization ability of the final phase compensation model can be evaluated using the test channel data. Using the test channel data that is independent of the model training process can effectively evaluate the generalization ability of the model and ensure the reliability of the phase compensation model.

In the ultrasonic imaging method provided in the embodiment of the present disclosure, the sample channel data is obtained, and the sample channel data includes training channel data and validation channel data. Then, the neural network model is trained using the training channel data to obtain an initial phase compensation model. Finally, model parameters of the initial phase compensation model are adjusted using the validation channel data to obtain the phase compensation model. In this method, an optional way to train the phase compensation model is provided. By dividing the sample channel data into the training channel data and the validation channel data, using the training channel data to train the neural network model, and using the validation channel data to fine-tune the trained neural network model, so that the phase compensation model is obtained.

The model is optimized through multiple iterations to minimize the difference between a prediction result of the model and an expected result. In an iteration process, the model parameters are adjusted based on the loss between the prediction result and the expected result. Based on this, a method for obtaining the initial phase compensation model is described in the following embodiment.

In some exemplary embodiments, as shown in FIG. 5, training the neural network model using the training channel data to obtain the initial phase compensation model includes the following steps of S501 to S503.

In S501, the training channel data is input into the neural network model to obtain training phase compensation data corresponding to the training channel data.

In some embodiments of the present disclosure, the training channel data is input into the neural network model, and the neural network model outputs the training phase compensation data corresponding to the training channel data.

In S502, a compensation loss is determined according to the training phase compensation data.

The compensation loss refers to a loss value determined according to an output result and a target result of the neural network model. In the embodiment of the present disclosure, it refers to a loss value determined by the training phase compensation data and the target phase compensation data of the training channel data. A phase of channel i in the target phase compensation data is defined as Ph(i). After obtaining the phase Ph(i), a compensation time

$$∇ τ ( i ) = \frac{P h ( i )}{2 π {f}_{c}}$$

can be obtained, where f_{c} refers to a center frequency of signal. Therefore, each delay of the channels is updated to τ(i)=τ₀(i)+∇τ(i) after phase compensation, where τ₀ refers to an initial delay of i-th channel calculated based on a geometric position of the ultrasonic probe and a sound propagation distance.

Exemplarily, the target phase compensation data corresponding to the training channel data is obtained, and then the compensation loss is determined according to a difference between the training phase compensation data and the target phase compensation data. Optionally, the compensation loss Loss may be determined according to the following formula (2):

$$Loss = {Σ}_{i = 1}^{n} ( {h}_{i} - {y}_{i} ) \qquad ( 2 )$$

where hᵢ refers to i-th target phase compensation data, yᵢ refers to i-th training phase compensation data, Loss refers to the compensation loss, a value range of i is 1 to n, i is a positive integer, n is the number of iterations.

In S503, the model parameters of the neural network model are adjusted based on the compensation loss until the compensation loss is less than a preset threshold, to obtain the initial phase compensation model.

When the compensation loss does not reach the preset threshold, it is necessary to continue adjusting the model parameters of the neural network model until the compensation loss is less than the preset threshold, then stop adjusting the model parameters to obtain the initial phase compensation model.

In the ultrasonic imaging method provided in the embodiment of the present disclosure, the training channel data is input into the neural network model to obtain the training phase compensation data corresponding to the training channel data, and then the compensation loss is determined according to the training phase compensation data. Finally, the model parameters of the neural network model are adjusted based on the compensation loss until the compensation loss is less than the preset threshold, to obtain the initial phase compensation model. In this method, parameters of the neural network model are adjusted according to the loss value between the output result and the expected result of the neural network model, so that a difference between an actual value and an ideal value of the neural network model is minimized, and the initial phase compensation model is obtained by training.

In the present disclosure, the neural network model is used to perform the phase compensation on the channel data. In order to evaluate the effect of this phase correction way and the quality of the ultimately reconstructed ultrasonic image, parameter values of relevant parameters representing phase distortion distribution can be used to evaluate the quality of the ultrasonic image. Based on this, in the following embodiment, a method for evaluating the quality of the ultrasonic image is described.

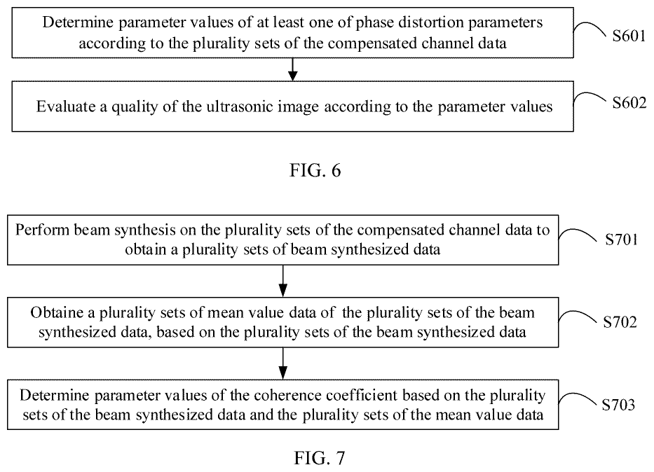

In some exemplary embodiments, as shown in FIG. 6, the method further includes the following steps of S601 and S602.

In S601, parameter values of at least one of phase distortion parameters are determined according to the plurality sets of the compensated channel data.

In some embodiments of the present disclosure, the phase distortion parameters include but are not limited to distortion intensity, correlation length of phase distortion, energy level fluctuation and coherence coefficient, etc.

The distortion intensity indicates severity of the phase aberration. In terms of image quality, greater distortion intensity indicates poorer image quality. The correlation length of the phase distortion is determined by a full width at half maximum (FWHM) of signal autocorrelation. As the correlation length of the phase distortion decreases, spatial frequency increases and aberration is introduced, resulting in greater image distortion. Greater energy level fluctuation, calculated as the root mean square of the RMS signal amplitude, indicates greater image distortion. Smaller coherence coefficient indicates greater image distortion.

Different phase distortion parameters have corresponding calculation methods. Based on this, for any phase distortion parameter, a calculation method corresponding to the phase distortion parameter is adopted, and parameter values of the phase distortion parameter are determined based on the plurality sets of the compensated channel data.

For example, for distortion intensity, firstly, beam synthesis is performed on the plurality sets of the compensated channel data to obtain a plurality sets of beam synthesized data, and then delay error data is obtained from the beam synthesized data, and then the root mean square of the delay error data is calculated to obtain the parameter values of the distortion intensity.

In S602, a quality of the ultrasonic image is evaluated according to the parameter values.

In some embodiments, the parameter values of the phase distortion parameters may be input into a pre-trained evaluation model, and the parameter values are analyzed by the evaluation model, to obtain an evaluation result of the ultrasonic image.

In other embodiments, each of the parameter values is compared with a corresponding preset parameter value threshold range. On a condition that each of the parameter values falls within the corresponding preset parameter value threshold range, it is determined that the quality of the ultrasonic image is qualified.

For any phase distortion parameter, there is a corresponding parameter value threshold range. On a condition that all parameter values are within the corresponding parameter value threshold range, the quality of the ultrasonic image is determined to be qualified. On a condition that at least one of the parameter values of the phase distortion parameters is not within the corresponding parameter value threshold range, the quality of the ultrasonic image is determined to be unqualified.

In the ultrasonic imaging method provided in the embodiment of the present disclosure, the parameter values of the at least one of the phase distortion parameters are determined according to the plurality sets of the compensated channel data, and then the quality of the ultrasonic image is evaluated according to the parameter values. In this method, a plurality sets of parameters related to phase distortion are used to evaluate the quality of the ultrasonic image. In this way, the quality of the ultrasonic image obtained after phase compensation through the neural network model is evaluated.

The coherence coefficient can also be used as a parameter for evaluating the quality of the ultrasonic image, and can be specifically determined based on the beam synthesized data of the compensated channel data. Based on this, in the following embodiment, a method for determining the parameter values of at least one of phase distortion parameters according to a plurality sets of the compensated channel data is described.

In some exemplary embodiments, as shown in FIG. 7, determining the parameter values of the at least one of the phase distortion parameters according to the plurality sets of the compensated channel data includes the following steps of S701 to S703.

In S701, beam synthesis is performed on the plurality sets of the compensated channel data to obtain a plurality sets of beam synthesized data.

The beam synthesis is performed on the plurality sets of the compensated channel data by a beam synthesis algorithm, to obtain a plurality sets of beam synthesized data. The beam synthesis algorithm may include but is not limited to beam synthesis using the delay superposition method and beam synthesis using the minimum variance method.

In S702, a plurality sets of mean value data of the plurality sets of the beam synthesized data are obtained, based on the plurality sets of the beam synthesized data.

For any set of the beam synthesized data, the mean value data of the beam synthesized data can be obtained by performing mean processing on the beam synthesized data.

In S703, parameter values of the coherence coefficient are determined based on the plurality sets of the beam synthesized data and the plurality sets of the mean value data.

After the plurality sets of mean value data of the plurality sets of the beam synthesized data are determined, the parameter values of the coherence coefficient can be determined based on the plurality sets of the beam synthesized data and the plurality sets of the mean value data. For example, the parameter values of the coherence coefficient can be determined according to the following formula (3):

$${C}_{m} = \frac{1}{N - m} {Σ}_{a = 1}^{a = N - m} \frac{{Σ}_{n = n 1}^{n = n 2} [ {S}_{a} ( n ) - avg ( {S}_{a} ) ] [ {S}_{a + m} ( n ) - a vg ( {S}_{b} ) ]}{\sqrt{{{Σ}_{n = n 1}^{n = n 2} [ {S}_{a} ( n ) - avg ( {S}_{a} ) ]}^{2} {{Σ}_{n = n 1}^{n = n 2} [ {S}_{a + m} ( n ) - avg ( {S}_{a + m} ) ]}^{2}}} \qquad ( 3 )$$

where Sₐ refers to beam synthesized data corresponding to channel a, S_{b} refers to beam synthesized data corresponding to channel b, m refers to a number of array elements between channels, a+m refers to a channel separated by m array elements from a, Sₐ₊ₘ refers to beam synthesized data corresponding to a channel separated by m array elements from channel a (i.e. channel a+m), avg(Sₐ) refers to mean value data of beam synthesized data corresponding to channel a, avg(S_{b}) refers to mean value data of beam synthesized data corresponding to channel b, avg(Sₐ₊ₘ) refers to mean value data of beam synthesized data corresponding to a channel separated by m array elements from channel a, n refers to a sampling point index corresponding to depth or time, n1 refers to number of starting sampling points, n2 refers to number of ending sampling points, n refers to an integer, and N refers to an integer, representing a number of all array elements in a transmit aperture.

In the ultrasonic imaging method provided in the embodiment of the present disclosure, the plurality sets of beam synthesized data are obtained by performing beam synthesis on the plurality sets of the compensated channel data, and then the plurality sets of the mean value data of the plurality sets of the beam synthesized data are obtained, based on the plurality sets of the beam synthesized data, and finally the parameter values of the coherence coefficient are determined based on the plurality sets of the beam synthesized data and the plurality sets of the mean value data. In this method, the beam synthesized data is obtained by performing beam synthesis on the plurality sets of the compensated channel data, and then the parameter values of the coherence coefficient are calculated and determined based on the beam synthesized data, which provides data support for evaluating the quality of the ultrasonic image.

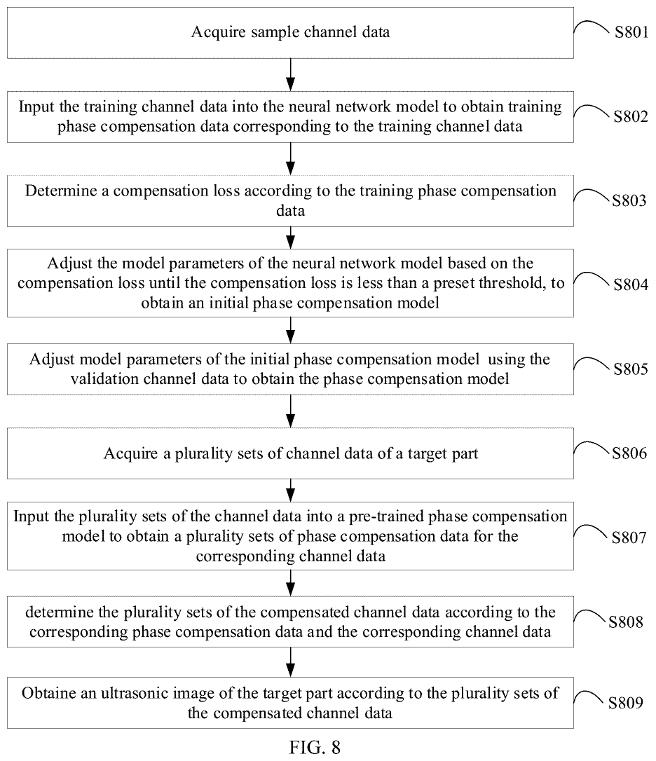

In addition, in some exemplary embodiments, an optional example of the ultrasonic imaging method is further provided in the present disclosure. As shown in FIG. 8, the method may include the following steps of S801 to S809.

In S801, sample channel data is acquired.

The sample channel data includes training channel data and validation channel data.

In S802, the training channel data is input into the neural network model to obtain training phase compensation data corresponding to the training channel data.

In S803, a compensation loss is determined according to the training phase compensation data.

In S804, the model parameters of the neural network model are adjusted based on the compensation loss until the compensation loss is less than a preset threshold, to obtain an initial phase compensation model.

In S805, model parameters of the initial phase compensation model are adjusted using the validation channel data to obtain the phase compensation model.

In S806, a plurality sets of channel data of a target part are acquired.

In S807, the plurality sets of the channel data are input into a pre-trained phase compensation model to obtain a plurality sets of phase compensation data for the corresponding channel data.

In S808, the plurality sets of the compensated channel data are determined according to the corresponding phase compensation data and the corresponding channel data.

In S809, an ultrasonic image of the target part is obtained according to the plurality sets of the compensated channel data.

The above process of S801 to S809 can refer to the description of the above method embodiment, and its implementation principle and technical effect are similar, which will not be repeated here.

It should be understood that although operations in flowcharts according to the embodiments described above are sequentially displayed according to an arrow, the operations may be not necessarily sequentially executed according to an order indicated by the arrow. Unless explicitly stated in the present disclosure, there is no strict order limit on an execution of the operations, and the operations may be executed in other orders. Moreover, at least some of the operations in flowcharts according to the embodiments described above may include a plurality of operations or a plurality of stages, the plurality of operations or the plurality of stages are not necessarily executed at the same time, but may be executed at different times, and the execution order of the plurality of operations or the plurality of stages is not necessarily sequential, but may be performed alternately with other operations or at least part of operations or stages in other operations.

Based on the same inventive concept, the embodiment of the present disclosure also provides an ultrasonic imaging apparatus to implement the ultrasonic imaging method involved above. An implementation solution for solving the problem provided by the device is similar to the implementation solution recorded in the above method, so the specific limitations in one or more embodiments of the ultrasonic imaging apparatus provided below can refer to the limitations on the ultrasonic imaging method above, and will not be repeated here.

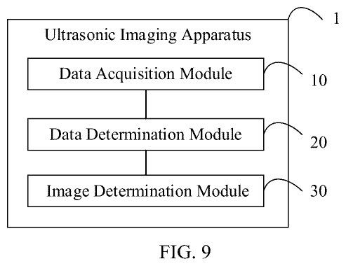

In some exemplary embodiments, as shown in FIG. 9, an ultrasonic imaging apparatus 1 is provided, including: a data acquisition module 10, a data determination module 20 and an image determination module 30.

The data acquisition module 10 is configured to acquire a plurality sets of channel data of a target part.

The data determination module 20 is configured to perform phase compensations on the plurality sets of the channel data to obtain a plurality sets of compensated channel data corresponding to the plurality sets of the channel data respectively.

The image determination module 30 is configured to obtain an ultrasonic image of the target part according to the plurality sets of the compensated channel data.

In some embodiments, the data determination module 20 is further configured to input the plurality sets of the channel data into a pre-trained phase compensation model to obtain a plurality sets of phase compensation data for the corresponding channel data, and determine each of the compensated channel data according to the corresponding phase compensation data and the corresponding channel data.

In some embodiments, the ultrasonic imaging apparatus 1 is further configured to acquire sample channel data, the sample channel data including training channel data and validation channel data, train a neural network model using the training channel data to obtain an initial phase compensation model, and adjust model parameters of the initial phase compensation model using the validation channel data to obtain the phase compensation model.

In some embodiments, the ultrasonic imaging apparatus 1 is further configured to input the training channel data into the neural network model to obtain training phase compensation data corresponding to the training channel data, determine a compensation loss according to the training phase compensation data, and adjust the model parameters of the neural network model based on the compensation loss until the compensation loss is less than a preset threshold, to obtain the initial phase compensation model.

In some embodiments, the ultrasonic imaging apparatus 1 is further configured to determine parameter values of at least one of phase distortion parameters according to the plurality sets of the compensated channel data, and evaluate the quality of the ultrasonic image according to the parameter values.

In some embodiments, the ultrasonic imaging apparatus 1 is further configured to perform beam synthesis on the plurality sets of the compensated channel data to obtain a plurality sets of beam synthesized data, obtain a plurality sets of mean value data of the plurality sets of the beam synthesized data based on the plurality sets of the beam synthesized data, and determine the parameter values of the coherence coefficient based on the plurality sets of the beam synthesized data and the plurality sets of the mean value data.

In some embodiments, the ultrasonic imaging apparatus 1 is further configured to compare each of the parameter values with a corresponding preset parameter value threshold range, and determine that the quality of the ultrasonic image is qualified on a condition that each of the parameter values falls within the corresponding preset parameter value threshold range.

The above-mentioned ultrasonic imaging apparatus can be implemented in whole or in part by software, hardware or a combination thereof. Each of the above-mentioned modules can be embedded in or independent of a processor in a computer device in the form of hardware, or can be stored in a memory in a computer device in the form of software, so that the processor can call and execute the operations corresponding to each of the above modules.

In some exemplary embodiments, a computer device is provided. The computer device includes a memory and a processor. A computer program is stored in the memory, and when the processor executes the computer program, the following steps are implemented: acquiring a plurality sets of channel data of a target part, performing phase compensations on the plurality sets of the channel data to obtain a plurality sets of compensated channel data corresponding to the plurality sets of the channel data respectively, and obtaining an ultrasonic image of the target part according to the plurality sets of the compensated channel data.

In some embodiments, when a step of performing the phase compensations on the plurality sets of the channel data to obtain the plurality sets of the compensated channel data corresponding to the plurality sets of the channel data in the computer program is performed by the processor, the following steps are specifically implemented: inputting the plurality sets of the channel data into a pre-trained phase compensation model to obtain a plurality sets of phase compensation data for the corresponding channel data, and determining the plurality sets of the compensated channel data according to the corresponding phase compensation data and the corresponding channel data.

In some embodiments, when a step of training the phase compensation model in the computer program is performed by the processor, the following steps are specifically implemented: acquiring sample channel data, the sample channel data including training channel data and validation channel data, training a neural network model using the training channel data to obtain an initial phase compensation model, and adjusting model parameters of the initial phase compensation model using the validation channel data to obtain the phase compensation model.

In some embodiments, when a step of training the neural network model using the training channel data to obtain the initial phase compensation model in the computer program is performed by the processor, the following steps are specifically implemented: inputting the training channel data into the neural network model to obtain training phase compensation data corresponding to the training channel data, determining a compensation loss according to the training phase compensation data, and adjusting the model parameters of the neural network model based on the compensation loss until the compensation loss is less than a preset threshold, to obtain the initial phase compensation model.

In some embodiments, when the computer program is performed by the processor, the following steps are further implemented: determining parameter values of at least one of phase distortion parameters according to the plurality sets of the compensated channel data, and evaluating the quality of the ultrasonic image according to the parameter values.

In some embodiments, when a step of determining the parameter values of the at least one of the phase distortion parameters according to the plurality sets of the compensated channel data in the computer program is performed by the processor, the following steps are specifically implemented: performing beam synthesis on the plurality sets of the compensated channel data to obtain a plurality sets of beam synthesized data, obtaining a plurality sets of mean value data of the plurality sets of the beam synthesized data, based on the plurality sets of the beam synthesized data, and determining the parameter values of the coherence coefficient based on the plurality sets of the beam synthesized data and the plurality sets of the mean value data.

In some embodiments, when a step of evaluating the quality of the ultrasonic image according to the parameter values in the computer program is performed by the processor, the following steps are specifically implemented: comparing each of the parameter values with a corresponding preset parameter value threshold range, and determining that the quality of the ultrasonic image is qualified on a condition that each of the parameter values falls within the corresponding preset parameter value threshold range.

The principles and specific processes of the computer device provided above in implementing each embodiment can be found in the description of the ultrasonic imaging method embodiment in the aforementioned embodiment, and will not be repeated here.

In some embodiments, a non-transitory computer-readable storage medium is provided, on which a computer program is stored. when the computer program is executed by a processor, the following steps are implemented: acquiring a plurality sets of channel data of a target part, performing phase compensations on the plurality sets of the channel data to obtain a plurality sets of compensated channel data corresponding to the plurality sets of the channel data respectively, and obtaining an ultrasonic image of the target part according to the plurality sets of the compensated channel data.

In some embodiments, when a step of performing the phase compensations on the plurality sets of the channel data to obtain the plurality sets of the compensated channel data corresponding to the plurality sets of the channel data in the computer program is performed by the processor, the following steps are specifically implemented: inputting the plurality sets of the channel data into a pre-trained phase compensation model to obtain a plurality sets of phase compensation data for the corresponding channel data, and determining the plurality sets of the compensated channel data according to the corresponding phase compensation data and the corresponding channel data.

In some embodiments, when a step of training the phase compensation model in the computer program is performed by the processor, the following steps are specifically implemented: acquiring sample channel data, the sample channel data including training channel data and validation channel data, training a neural network model using the training channel data to obtain an initial phase compensation model, and adjusting model parameters of the initial phase compensation model using the validation channel data to obtain the phase compensation model.

In some embodiments, when a step of training the neural network model using the training channel data to obtain the initial phase compensation model in the computer program is performed by the processor, the following steps are specifically implemented: inputting the training channel data into the neural network model to obtain training phase compensation data corresponding to the training channel data, determining a compensation loss according to the training phase compensation data, and adjusting the model parameters of the neural network model based on the compensation loss until the compensation loss is less than a preset threshold, to obtain the initial phase compensation model.

In some embodiments, when the computer program is performed by the processor, the following steps are further implemented: determining parameter values of at least one of phase distortion parameters according to the plurality sets of the compensated channel data, and evaluating the quality of the ultrasonic image according to the parameter values.

In some embodiments, when a step of determining the parameter values of the at least one of the phase distortion parameters according to the plurality sets of the compensated channel data in the computer program is performed by the processor, the following steps are specifically implemented: performing beam synthesis on the plurality sets of the compensated channel data to obtain a plurality sets of beam synthesized data, obtaining a plurality sets of mean value data of the plurality sets of the beam synthesized data, based on the plurality sets of the beam synthesized data, and determining the parameter values of the coherence coefficient based on the plurality sets of the beam synthesized data and the plurality sets of the mean value data.

In some embodiments, when a step of evaluating the quality of the ultrasonic image according to the parameter values in the computer program is performed by the processor, the following steps are specifically implemented: comparing each of the parameter values with a corresponding preset parameter value threshold range, and determining that the quality of the ultrasonic image is qualified on a condition that each of the parameter values falls within the corresponding preset parameter value threshold range.

The principles and specific processes of the computer-readable storage medium provided above in implementing each embodiment can be found in the description of the ultrasonic imaging method embodiment in the aforementioned embodiment, and will not be repeated here.

In some embodiments, a computer program product is provided, including a computer program. When the computer program executed by a processor, the following steps are specifically implemented: acquiring a plurality sets of channel data of a target part, performing phase compensations on the plurality sets of the channel data to obtain a plurality sets of compensated channel data corresponding to the plurality sets of the channel data respectively, and obtaining an ultrasonic image of the target part according to the plurality sets of the compensated channel data.

In some embodiments, when a step of performing the phase compensations on the plurality sets of the channel data to obtain the plurality sets of the compensated channel data corresponding to the plurality sets of the channel data in the computer program is performed by the processor, the following steps are specifically implemented: inputting the plurality sets of the channel data into a pre-trained phase compensation model to obtain a plurality sets of phase compensation data for the corresponding channel data, and determining the plurality sets of the compensated channel data according to the corresponding phase compensation data and the corresponding channel data.

In some embodiments, when a step of training the phase compensation model in the computer program is performed by the processor, the following steps are specifically implemented: acquiring sample channel data, the sample channel data including training channel data and validation channel data, training a neural network model using the training channel data to obtain an initial phase compensation model, and adjusting model parameters of the initial phase compensation model using the validation channel data to obtain the phase compensation model.

In some embodiments, when a step of training the neural network model using the training channel data to obtain the initial phase compensation model in the computer program is performed by the processor, the following steps are specifically implemented: inputting the training channel data into the neural network model to obtain training phase compensation data corresponding to the training channel data, determining a compensation loss according to the training phase compensation data, and adjusting the model parameters of the neural network model based on the compensation loss until the compensation loss is less than a preset threshold, to obtain the initial phase compensation model.

In some embodiments, when the computer program is performed by the processor, the following steps are further implemented: determining parameter values of at least one of phase distortion parameters according to the plurality sets of the compensated channel data, and evaluating a quality of the ultrasonic image according to the parameter values.

In some embodiments, when a step of determining the parameter values of the at least one of the phase distortion parameters according to the plurality sets of the compensated channel data in the computer program is performed by the processor, the following steps are specifically implemented: performing beam synthesis on the plurality sets of the compensated channel data to obtain a plurality sets of beam synthesized data, obtaining a plurality sets of mean value data of the plurality sets of the beam synthesized data, based on the plurality sets of the beam synthesized data, and determining the parameter values of the coherence coefficient based on the plurality sets of the beam synthesized data and the plurality sets of the mean value data.

In some embodiments, when a step of evaluating the quality of the ultrasonic image according to the parameter values in the computer program is performed by the processor, the following steps are specifically implemented: comparing each of the parameter values with a corresponding preset parameter value threshold range; and determining that the quality of the ultrasonic image is qualified on a condition that each of the parameter values falls within the corresponding preset parameter value threshold range.

The principles and specific processes of the computer program product provided above in implementing each embodiment can be found in the description of the ultrasonic imaging method embodiment in the aforementioned embodiment, and will not be repeated here.

It should be noted that the data (including but not limited to data used for analysis, stored data, displayed data, etc.) involved in the present disclosure are those authorized by the user or sufficiently authorized by the parties. The collection, use and processing of the relevant data need to comply with relevant regulations.

Those of ordinary skill in the art can understand that all or part of the processes in the methods of the above embodiments can be completed by instructing relevant hardware through a computer program. The computer program can be stored in a non-transitory computer-readable storage. In the media, when executed, the computer program may include the processes of the above method embodiments. Any reference to memory, database or other media used in the embodiments provided in the present disclosure may include at least one of non-transitory and transitory memory. Non-transitory memory can include read-only memory (ROM), magnetic tape, floppy disk, flash memory, optical memory, high-density embedded non-transitory memory, resistive memory (ReRAM), magnetic variable memory (Magnetoresistive Random Access Memory (MRAM), ferroelectric memory (Ferroelectric Random Access Memory, FRAM), phase change memory (Phase Change Memory, PCM), graphene memory, etc. Transitory memory may include random access memory (Random Access Memory, RAM) or external cache memory, etc. By way of illustration and not limitation, RAM can be in many forms, such as static random access memory (Static Random Access Memory, SRAM) or dynamic random access memory (Dynamic Random Access Memory, DRAM). The databases involved in the various embodiments provided in the present disclosure may include at least one of a relational database and a non-relational database. Non-relational databases may include blockchain-based distributed databases, etc., but are not limited thereto. The processors involved in the various embodiments provided in the present disclosure may be general-purpose processors, central processing units, graphics processors, digital signal processors, programmable logic devices, quantum computing-based data processing logic devices, etc., and are not limited to this.

The various technical features of the above embodiments can be combined arbitrarily, and in order to make the description concise, all possible combinations of the various technical features of the above embodiments have not been described; however, as long as there is no contradiction in the combinations of these technical features, all of them should be considered to be within the scope of the present specification.

The above-described embodiments express only several embodiments of the present disclosure, which are described in a more specific and detailed manner, but are not to be construed as a limitation of the scope of the patent of the present disclosure. It should be pointed out that, for a person of ordinary skill in the art, several deformations and improvements can be made without departing from the conception of the present disclosure, all of which fall within the scope of protection of the present disclosure. Therefore, the scope of protection of this disclosure shall be subject to the attached claims.

What is claimed is:

1. An ultrasonic imaging method, comprising:
acquiring a plurality sets of channel data of a target part;
performing phase compensations on the plurality sets of the channel data to obtain a plurality sets of compensated channel data corresponding to the plurality sets of the channel data respectively; and
obtaining an ultrasonic image of the target part according to the plurality sets of the compensated channel data.

2. The ultrasonic imaging method according to claim 1, wherein performing the phase compensations on the plurality sets of the channel data to obtain the plurality sets of the compensated channel data corresponding to the plurality sets of the channel data respectively comprises:
inputting the plurality sets of the channel data into a pre-trained phase compensation model to obtain a plurality sets of phase compensation data for the corresponding channel data; and
determining the plurality sets of the compensated channel data according to the corresponding phase compensation data and the corresponding channel data.

3. The ultrasonic imaging method according to claim 2, further comprising training the phase compensation model, wherein training the phase compensation model comprises:
acquiring sample channel data, the sample channel data comprising training channel data and validation channel data;
training a neural network model using the training channel data to obtain an initial phase compensation model; and
adjusting model parameters of the initial phase compensation model using the validation channel data to obtain the phase compensation model.

4. The ultrasonic imaging method according to claim 3, wherein the sample channel data further comprises test channel data, and the ultrasonic imaging method further comprises:
evaluating a generalization ability of the phase compensation model based on the test channel data.

5. The ultrasonic imaging method according to claim 3, wherein training the neural network model using the training channel data to obtain the initial phase compensation model comprises:
inputting the training channel data into the neural network model to obtain training phase compensation data corresponding to the training channel data;
determining a compensation loss according to the training phase compensation data; and
adjusting the model parameters of the neural network model based on the compensation loss until the compensation loss is less than a preset threshold, to obtain the initial phase compensation model.

6. The ultrasonic imaging method according to claim 5, wherein determining the compensation loss according to the training phase compensation data comprises:
acquiring target phase compensation data corresponding to the training channel data; and
determining the compensation loss according to the training phase compensation data and the target phase compensation data.

7. The ultrasonic imaging method according to claim 1, wherein the ultrasonic imaging method further comprises:
determining parameter values of at least one of phase distortion parameters according to the plurality sets of the compensated channel data; and
evaluating a quality of the ultrasonic image according to the parameter values.

8. The ultrasonic imaging method according to claim 7, wherein the phase distortion parameters comprise at least one of distortion intensity, correlation length of phase distortion, energy level fluctuation, and coherence coefficient.

9. The ultrasonic imaging method according to claim 7, wherein the phase distortion parameters comprise a coherence coefficient, and determining the parameter values of the at least one of the phase distortion parameters according to the plurality sets of the compensated channel data comprises:
performing beam synthesis on the plurality sets of the compensated channel data to obtain a plurality sets of beam synthesized data;
obtaining a plurality sets of mean value data of the plurality sets of the beam synthesized data based on the plurality sets of the beam synthesized data;
determining the parameter values of the coherence coefficient based on the plurality sets of the beam synthesized data and the plurality sets of the mean value data.

10. The ultrasonic imaging method according to claim 7, wherein the phase distortion parameters comprise distortion intensity, and determining the parameter values of the at least one of the phase distortion parameters according to the plurality sets of the compensated channel data comprises:
performing beam synthesis on the plurality of the compensated channel data to obtain a plurality sets of beam synthesized data; and
acquiring delay error data from the beam synthesized data, and determining parameter values of the distortion intensity based on the delay error data.

11. The ultrasonic imaging method according to claim 7, wherein evaluating the quality of the ultrasonic image according to the parameter values comprises:
comparing each of the parameter values with a corresponding preset parameter value threshold range; and
determining that the quality of the ultrasonic image is qualified on a condition that each of the parameter values falls within the corresponding preset parameter value threshold range.

12. The ultrasonic imaging method according to claim 1, wherein obtaining the ultrasonic image of the target part according to the plurality sets of the compensated channel data comprises:
performing beam synthesis on the plurality sets of the compensated channel data to obtain image data; and
obtaining the ultrasonic image according to the image data.

13. A computer device comprising a memory and a processor, wherein the memory stores a computer program, and the processor, when executing the computer program, implements:
acquiring a plurality sets of channel data of a target part;
performing phase compensations on the plurality sets of the channel data to obtain a plurality sets of compensated channel data corresponding to the plurality sets of the channel data respectively; and
obtaining an ultrasonic image of the target part according to the plurality sets of the compensated channel data.

14. The computer device according to claim 13, wherein performing the phase compensations on the plurality sets of the channel data to obtain the plurality sets of the compensated channel data corresponding to the plurality sets of the channel data comprises:
inputting the plurality sets of the channel data into a pre-trained phase compensation model to obtain a plurality sets of phase compensation data for the corresponding channel data; and
determining the plurality sets of the compensated channel data according to the corresponding phase compensation data and the corresponding channel data.

15. The computer device according to claim 14, wherein the processor further implements training the phase compensation model, and training the phase compensation model comprises:
acquiring sample channel data, the sample channel data comprising training channel data and validation channel data;
training a neural network model using the training channel data to obtain an initial phase compensation model; and
adjusting model parameters of the initial phase compensation model using the validation channel data to obtain the phase compensation model.

16. The computer device according to claim 15, wherein training the neural network model using the training channel data to obtain the initial phase compensation model comprises:
inputting the training channel data into the neural network model to obtain training phase compensation data corresponding to the training channel data;
determining a compensation loss according to the training phase compensation data; and
adjusting the model parameters of the neural network model based on the compensation loss until the compensation loss is less than a preset threshold, to obtain the initial phase compensation model.

17. The computer device according to claim 13, wherein the processor further implements:
determining parameter values of at least one of phase distortion parameters according to the plurality of the compensated channel data; and
evaluating a quality of the ultrasonic image according to the parameter values.

18. The computer device according to claim 17, wherein determining the parameter values of the at least one of phase distortion parameters according to the plurality of the compensated channel data comprises:
performing beam synthesis on the plurality sets of the compensated channel data to obtain a plurality sets of beam synthesized data;
obtaining a plurality sets of mean value data of the plurality sets of the beam synthesized data, based on the plurality sets of the beam synthesized data; and
determining parameter values of coherence coefficient based on the plurality sets of the beam synthesized data and the plurality sets of the mean value data, the phase distortion parameters comprising the coherence coefficient.

19. The computer device according to claim 17, wherein evaluating the quality of the ultrasonic image according to the parameter values comprises:
comparing each of the parameter values with a corresponding preset parameter value threshold range; and
determining that the quality of the ultrasonic image is qualified on a condition that each of the parameter values falls within the corresponding preset parameter value threshold range.

20. A non-transitory computer-readable storage medium having a computer program stored thereon, wherein the computer program, when executed by a processor, causes the processor to perform an ultrasonic imaging method, the ultrasonic imaging method comprising:
acquiring a plurality sets of channel data of a target part;
performing phase compensations on the plurality sets of the channel data to obtain a plurality sets of compensated channel data corresponding to the plurality sets of the channel data respectively; and
obtaining an ultrasonic image of the target part according to the plurality sets of the compensated channel data.
